(19) United States
(12) Patent Application Publication
(10) Pub. No.: US 2004/0248829 A1
(43) Pub. Date: Dec. 9, 2004

(54) Functions for d52 and ra006 nucleic acids and polypeptides

(76) Inventor: Cord Dohrmann, Gottingen (DE)

Correspondence Address: Sutherland Assill & Brennan, 999 Peachtree Street NE, Atlanta, GA, 30309-3996, US

(21) Appl. No.: 10/477,987

(22) PCT Filed: May 13, 2002
(86) PCT No.: PCT/EP02/05243

(30) Foreign Application Priority Data
May 11, 2001 (EP) 01111575.5
May 22, 2001 (EP) 01112462.5

(51) Int. Cl. A61K 48/00; A61K 39/395

(52) U.S. Cl. 514/44; 424/146.1; 514/12

(57) ABSTRACT

The present invention discloses polynucleotides which identify and encode pD52 or pRA006 as well as novel functions for the D52 gene family (pD52) which are specifically expressed in the embryonic pancreatic epithelium, in islet cells of the pancreas, in adipocytes, and in intestinal crypt cells. The invention also discloses novel function for the RA006 gene family (pRA006) which are specifically expressed in a subset of islet cells of the pancreas, the eye, as well as the central and peripheral nervous system. The invention provides for compositions for disorders associated with the expression of pD52 or pRA006, such as for the treatment, alleviation and/or prevention of metabolic disorder or metabolic syndrome, such as pancreatic dysfunction (for example diabetes, hyperglycemia, and impaired glucose tolerance), obesity, adipositas, eating disorders (bulimia nervosa, anorexia nervosa), cachexia (wasting), and neurodegenerative disorders, and other diseases and disorders.

### DESCRIPTION

This invention relates to nucleic acid sequences of the D52 gene family and polypeptides encoded thereby, specifically expressed in the embryonic pancreatic epithelium, in islet cells of the pancreas, in adipocytes, and in intestinal crypt cells, and to nucleic acid sequences of the RA006 gene family and polypeptides encoded thereby, specifically expressed in a subset of islet cells of the pancreas, the eye, as well as the central and peripheral nervous system. The invention relates to the use of these nucleic acids and polypeptides in the diagnosis, study, prevention, and treatment of diseases and disorders, for example, but not limited to, of the pancreas including metabolic disorders such as diabetes, adipositas, and neurodegenerative diseases and others.

The protein described in this invention shows significant homologies to human D52 protein as described in, but not limited to, the references below:

Members of the mammalian D52 protein family are known as signalling molecules which act as regulators of cell proliferation. Human D52 (N8 protein, Genbank Accession Number S82081) was found to be overexpressed in breast and lung carcinomas (see, for example, Byrne et al., 1995, Cancer Res. 55:2896-2903 and Chen et al., 1996, Oncogene 12(4):741-51). Highly conserved orthologues of the human D52 have been cloned from mouse (Genbank Accession Number U44426), rabbit (SPTREMBL-Accession Number Q95212), and Japanese quail (Genbank Accession Number Y07757). The rabbit D52 homologue CSPP28 (SPTREMBL-Accession Number Q95212) is a calcium sensitive phospho-protein of 28 kDA, which is phosphorylated in response to cholinergic stimulation of gastric pareital cell (see, for example, Parente et al., 1996, J. Biol. Chem. 271:20096-20101). In the Japanese quail, a D52 homologue R10, a cytoplasmic protein of 23 kDa, was detected in several embryonic tissues. The R10 protein contains a leucine zipper-containing region at its amino-terminal end, and can thereby readily form homodimers (see, for example, Proux et al., 1996, 271(48):30790-30797). Generally, the D52 protein family members have characteristic highly conserved coiled-coil domains suggesting functions involving protein-protein interactions and amino- and carboxy-terminally located PEST domains (see, for example, Byrne et al., 1996, Genomics 35(3):523-532). Human breast carcinoma-associated D53 proteins are predicted to be 52% identical to human D52. In situ hybridizations of breast carcinoma sections have shown that the D52 gene was expressed in cancer cells, especially in breast and lung carcinomas (see, for example, Byrne et al., 1995, Cancer Res. 55(13):2896-2903, 2903, and Chen et al., 1996, Oncogene 12(4):741-51). The cloning of human D54 protein indicates alternative coding sequence usage in D52-like transcripts. The alternative splicing within the D52 gene family functions in part to alter the protein-protein interaction capabilities of encoded isoforms (see, for example, Nourse et al., 1998, Biochim. Biophys. Acta 1443(1-2):1.55-168; International Application WO99/51379).

Although a link of D52 protein family member has been found to several types of cancer and malignomas, a function in the regulation of metabolic diseases such as obesity and diabetes has not been described in the prior art. Interestingly, it has been found that the expression of mouse D52 is 5.2-fold upregulated in the adipose tissue of obese mice, see for example Nadler et. al., 2000, Proc. Natl. Acad. Sci. U.S.A. 97(21):11371-11376. This invention describes novel functions for the D52 gene family that is involved in the development of the pancreas, differentiation of adipocytes, and could be used as a marker for intestinal stem cells.

The identification of polynucleotides encoding molecules specifically expressed in the embryonic pancreatic epithelium, in islet cells of the pancreas, and in intestinal crypt cells, and the molecules themselves, presents the opportunity to investigate and/or modulate diseases and disorders of the pancreas, including diabetes. The identification of molecules related to the D52 protein satisfies a need in the art by providing new compositions useful in diagnosis, treatment, and prognosis of pancreatic diseases, adipositas and other metabolic disorders.

The RA006 protein described in this invention shows significant homologies to a hypothetical human protein (Genbank Accession Number CAB53688) which was isolated from fetal kidney tissue. A function in the regulation of diseases and disorders, for example, but not limited to, of the pancreas (such as diabetes), and related diseases and disorders (such as adipositas), and neurodegenerative diseases has not been described in the prior art. This invention describes novel functions for the RA006 gene family that is involved in the development of the pancreas, the eye, as well as the central and peripheral nervous.

The identification of polynucleotides encoding molecules specifically expressed in a subset of islet cells of the pancreas, the eye, as well as the central and peripheral nervous system presents the opportunity to investigate diseases and disorders of the pancreas including diabetes and obesity. Furthermore, it presents the opportunity to investigate diseases and disorders affecting the eye as well as the central and peripheral nervous. The identification of molecules related to the RA006 protein satisfies a need in the art by providing new compositions useful in diagnosis, treatment, and prognosis of diseases and disorders, for example, but not limited to, of the pancreas (such as diabetes), and related diseases and disorders (such as adipositas), and neurodegenerative diseases.

### SUMMARY OF THE INVENTION

The present invention features a D52-like protein specifically expressed in the embryonic pancreatic epithelium, in islet cells of the pancreas, in adipocytes, and in intestinal crypt cells herein after designated pD52 and characterized as having similarity to proteins of the D52 protein family. The present invention also features a RA006-like protein specifically expressed in a subset of islet cells of the pancreas, the eye, as well as the central and peripheral nervous system and characterized as having similarity to a hypothetical human protein.

D52 or RA006 proteins and nucleic acid molecules coding therefor are obtainable from vertebrate species, e.g. mammals or birds. Particularly preferred are human D52 nucleic acids (Genbank Acc. No. S82081 or AAB 36476) or human RA006-homolog nucleic acids or polypeptides encoded thereby (GenBank Acc. No. CAB 53688). Also particularly preferred are chicken D52 (FIG. 1; SEQ ID NO: 1 and 2) or RA006 (FIG. 6; SEQ ID NO: 3 and 4) nucleic acids and polypeptides encoded thereby. Accordingly, the invention features a substantially purified pD52 or RA006 polypeptide which has the amino acid sequence shown in SEQ ID NO:2 or 4, respectively. One aspect of the invention features isolated and substantially purified polynucleotides that encode pD52 or pRA006. In a particular aspect, the polynucleotide is the nucleotide sequence of SEQ ID NO:1 or SEQ ID NO:3. The invention also relates to a polynucleotide sequence comprising the complement of SEQ ID NO:1 or SEQ ID NO:3 or variants thereof. In addition, the invention features polynucleotide sequences which hybridize under stringent conditions to SEQ ID NO:1 or SEQ ID NO:3. The invention additionally features nucleic acid sequences including polypeptides, oligonucleotides, peptide nucleic acids (PNA), morpholinonucleic acids, locked nucleic acids (LNA), fragments, portions or antisense molecules thereof, and expression vectors and host cells comprising polynucleotides that encode pD52 or pRA006. The present invention also features antibodies which bind specifically to pD52 or pRA006, and pharmaceutical compositions comprising substantially purified pD52 or pRA006. The invention also features the use of agonists and antagonists of pD52 or pRA006.

### BRIEF DESCRIPTION OF THE FIGURES

FIG. 1: Chicken D52

FIG. 1A: Nucleic acid sequence (SEQ ID NO:1) encoding the D52-like protein of chicken D52. 5′ and 3′ untranslated regions are underlined and the start and stop codons are in bold font.

FIG. 1B: Protein sequence (SEQ ID NO:2) encoded by the coding sequence shown in FIG. 1A. The coiled-coil domain predicted in the chick D52-like protein is in bold font.

FIG. 2: Amino acid sequence alignments among the pD52 protein of the invention (line 2; SEQ ID NO:2), human tumor protein D52 (N8-tumor expression-enhanced gene) (line 4, SWISSPROT-Accession Number P55327, identical to GenBank Accession Number S82081; the human tumor protein D52 is identical to the first 184 amino acids of the human N8 protein long isoform, 248 amino acids, SPTREMBL-Accession Number Q9UCX8, not shown) mouse mD52 protein (line 3, SPTREMBL-Accession Number Q62393, identical to GenBank Accession Number U44426), and Japanese quail (C. coturnix japonica) N10 protein (line 1, SPTREMBL-Accession Number P79700). The alignment was produced using the multisequence alignment program of ClustalW (1.7) software (Thompson, J. D., 1997, Nucleic Acids Research, 24:4876-4882).

FIG. 3: In situ hybridization results for the pD52-like protein of the invention. FIG. 3a, b, c show whole-mount in situ hybridizatons on chick embryos (day 5). lb 32 lung bud, pb=pancreatic bud, m=mesonephros, li=liver anlage, dpb=dorsal pancreatic bud, vbp=ventral pancreatic bud; FIG. 3d and e show in situ hybridizations on pancreatic bud tissue sections. pe=pancreatic epithelium, du=duodenum, is=islet.

FIG. 4: In situ hybridization results for the pD52-like protein of the invention. FIG. 4a—human duodenum, arrows point to crypt cells. FIG. 4b, human pancreas, is=islets.

FIG. 5: RT-PCR analysis of the pD52-like protein of the invention in various preadipocyte cell lines. FIG. 5A shows in Lane 1, Molecular Weight Marker, Lane 2, No template control (Con), Lane 3, NIH3T3 (normal fibroblast), Lanes 4-7, preadipoctes, Lane 4, 3T3-F442A, Lane 5, 3T3-L1, Lane 6, TA1, Lane 7, HIB-IB), and Lane 8, Molecular Weight Marker. Lower panel depicts same samples analyzed for actin to control for the integrity of the cDNAs in each sample.

FIG. 6a: Nucleic acid sequence (SEQ ID NO:3) encoding the RA006-like protein of chicken RA006. 5′ and 3′ untranslated regions are underlined and the start and stop codons are in bold font. FIG. 6B: Protein sequence (SEQ ID NO:4) encoded by the coding sequence shown in FIG. 6A.

FIG. 7: Amino acid sequence alignment among the pRA006 protein of the invention (line 1; SEQ ID NO:4) and a hypothetical human protein (Genbank Accession Number CAB53688; SPTREMBL-Accession Number Q9UFZ0)). Shown is the BlastP search result. Reference: Altschul et al., 1990, Basic local alignment search too, J. Mol. Biol. 215:403-410)

FIG. 8: In situ hybridization results for the RA006-like protein of the invention. FIG. 8A, B, C show whole-mount in situ hybridizatons on chick embryos (A: day 4; B and C: day 5). FIG. 8D, E, and F show in situ hybridizations on pancreatic bud tissue sections.

### DESCRIPTION OF THE INVENTION

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention.

The Invention

The invention is based on the identification of novel functions for D52-like and RA006-like proteins. It is described in this invention that pD52 protein is specifically expressed in the embryonic pancreatic epithelium, in islet cells of the pancreas, in adipocytes, and marks intestinal crypt cells, and that pRA006 protein is specifically expressed in a subset of islet cells of the pancreas, the eye, as well as the central and peripheral nervous system. The invention is based on polynucleotides encoding pD52 or pRA006 and compositions comprising the polynucleotides and/or polypeptides, and the use of these compositions for the diagnosis, study, prevention, or treatment of diseases and disorders related to such cells, including metabolic diseases, e.g. disorders associated with the fat and/or carbohydrate metabolism, such as diabetes and obesity, and neurodegenerative disorders, and others.

Nucleic acids encoding the chicken pD52 or pRA006 of the present invention were first identified in DeveloGen Clones RA290 and RA006, respectively, from a pancreas tissue cDNA library (day 6) through a whole-mount in situ screen for genes expressed in the embryonic pancreatic bud.

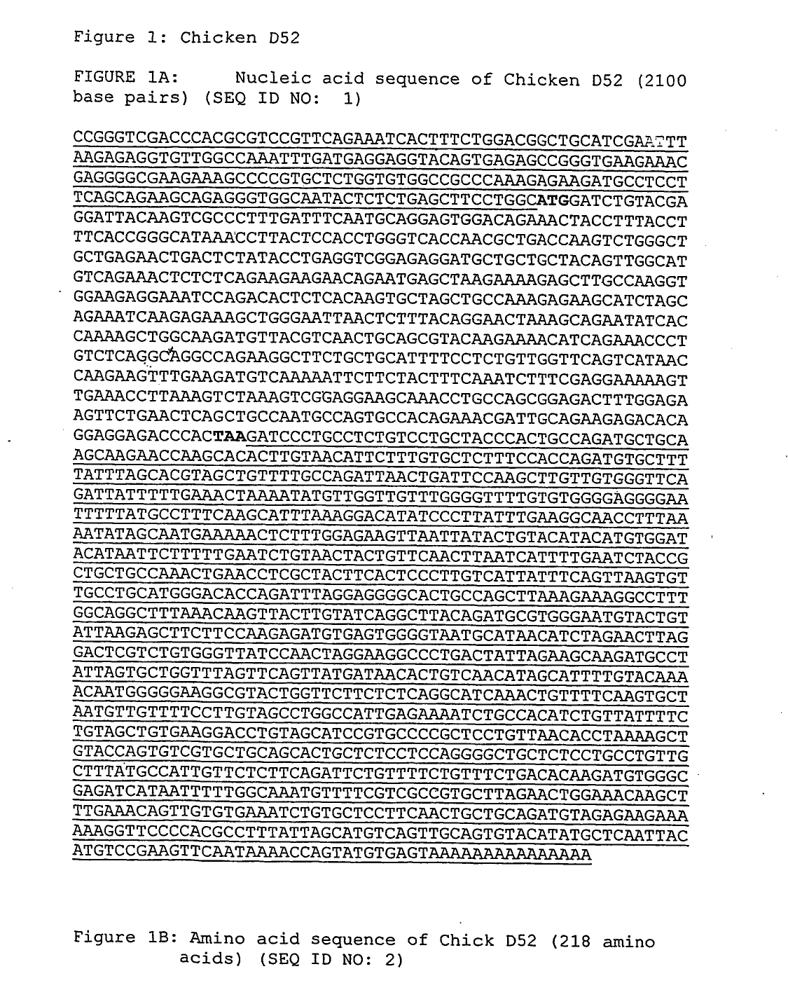

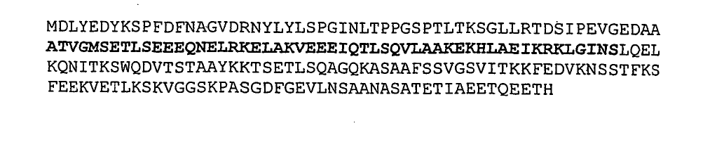

In one embodiment, the invention encompasses the D52-like protein specifically expressed in the embryonic pancreatic epithelium, in islet cells of the pancreas, in adipocytes, and in intestinal crypt cells, a polypeptide comprising the amino acid sequence of SEQ ID NO:2, as presented using the one-letter code in FIG. 1B. pD52 is 218 amino acids in length. An open reading was identified beginning with an ATP initiation codon at nucleotide 221 and ending with a TAA stop codon at nucleotide 874 (shown in bold in FIG. 1A). Putative untranslated regions upstream from the initiation codon and downstream from the termination codon are underlined in FIG. 1. The calculated molecular weight of the protein of the invention is 23662.2 daltons, and the theoretical pl is 4.89.

The predicted amino acid sequence was searched in the publicly available GenBank database. In search of sequence databases, it was found, for example, that pD52 has homology with Japanese quail (Coturnix coturnix japonica) R10 protein (SPTREMBL- Accession Number: P79700; GenBank Accession Number Y07757) and human D52 (N8 protein; SWISSPROT-Accession Number: P55327; GenBank Accession Number S82081)). In particular, pD52 and Japanese quail R10 protein share 94% identity, and pD52 and human protein D52 (N8 tumor expression-enhanced gene) share 84% identity starting between amino acid 40 and 215 of pD52. In addition, sequences homologous to D52 have been identified in Zebrafish (Danio, Genbank accession number BE558124), Caenorhabditis elegans (Genbank accession number P55326), and Drosophila melanogaster (Genbank accession number AAF57690).

Based upon homology, pD52 protein and each homologous protein or peptide may share at least some activity.

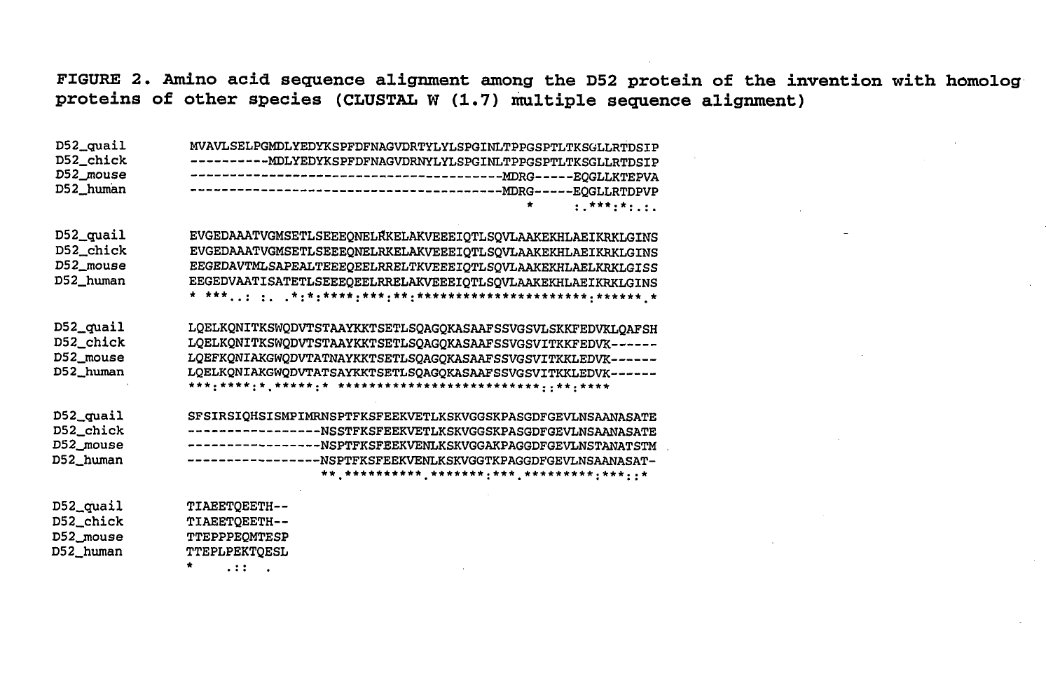

A multiple sequence aligment is given in FIG. 2 with the protein of the invention being shown on line 4, in a ClustalW (1.7) analysis comparing the protein of the invention (line 2, D52_chick) with related protein sequences from different species, such as the human D52 protein (line 4, labeled ‘D52_human’, mouse (line 3, ‘D52_mouse’), and quail (line 1, ‘D52_quail’).

The protein of the invention was searched against other databases using SignalPep and PSort search protocols. The D52 protein of the invention is most likely located in the cytoplasm and seems to have no aminoterminal signal sequence.

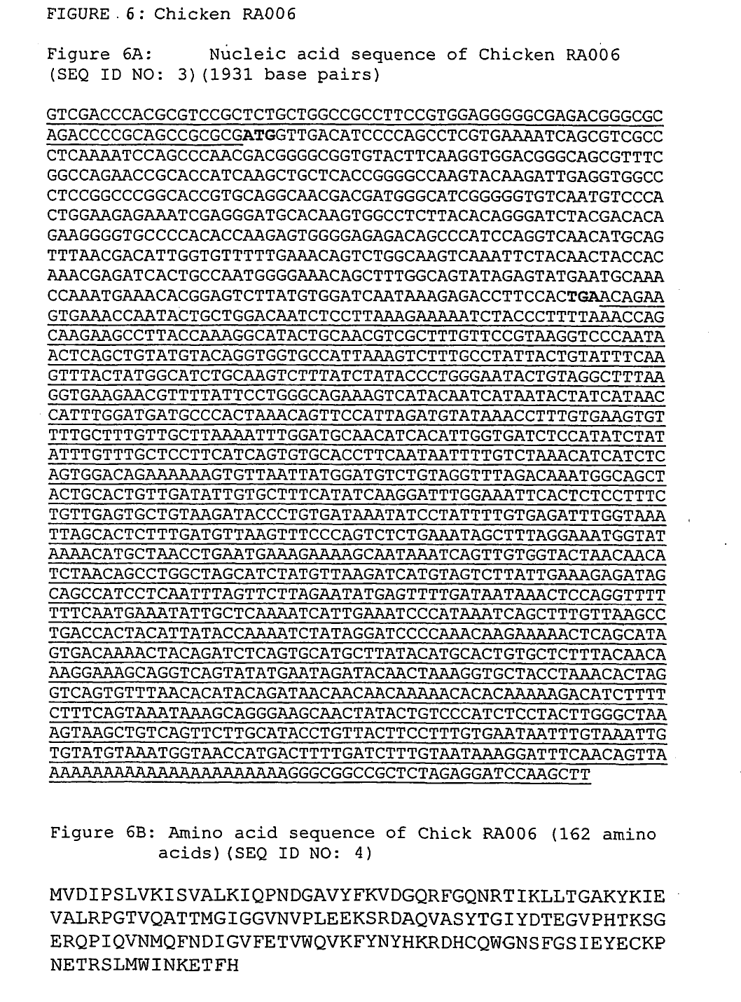

In a further embodiment, the invention encompasses the RA006-like protein specifically in a subset of islet cells of the pancreas, the eye, as well as the central and peripheral nervous system, a polypeptide comprising the amino acid sequence of SEQ ID NO:4, as presented using the one-letter code in FIG. 6B. pRA006 is 162 amino acids in length. An open reading was identified beginning with an ATP initiation codon and ending with a TGA stop codon (shown in FIG. 6A). The calculated molecular weight of the protein of the invention is 18373.89 daltons, and the theoretical pl is 8.49.

The predicted amino acid sequence was searched in the publicly available GenBank database. In search of sequence databases, it was found, for example, that pRA006 has homology with a hypothetical human protein (Genbank Accession Number CAB53688) as well as a human EST (Genbank Accession Number AL536254), a mouse EST (Genbank Accession Number AA048465), a zebrafish EST (Genbank Accession Number AW280893), a bovine EST (Genbank Accession Number BE479822). Based upon homology, pRA006 protein and each homologous protein or peptide may share at least some activity.

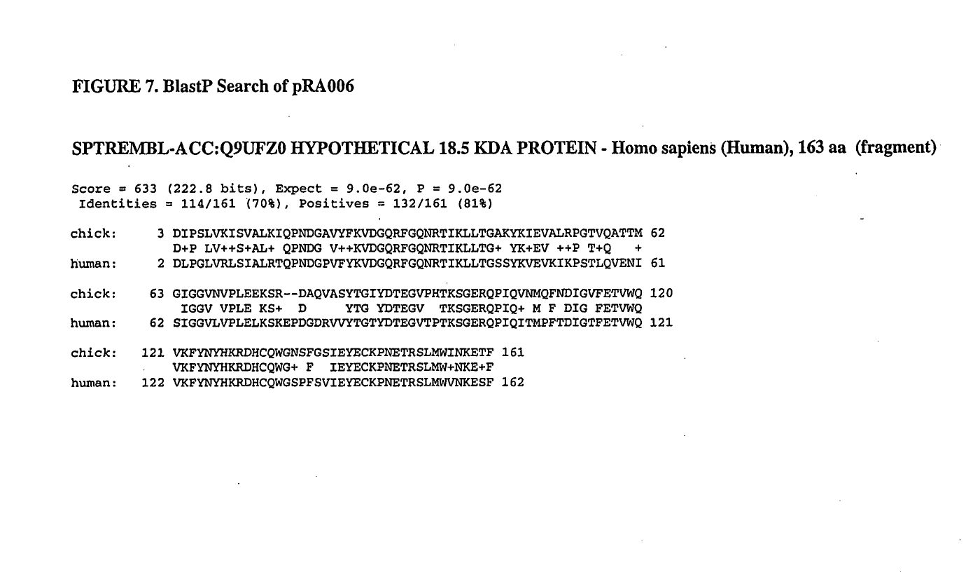

The result of a BlastP search is shown in FIG. 7 comparing the protein of the invention (‘chick’) with a related human protein sequence (‘human’). The human hypothetical 18.5 kDa protein is 81% homolog to the chicken RA006 protein.

The protein of the invention was searched against other databases using SignalPep and PSort search protocols. The RA006 protein of the invention is most likely located in the cytoplasm and seems to have no N-terminal signal sequence.

Expression

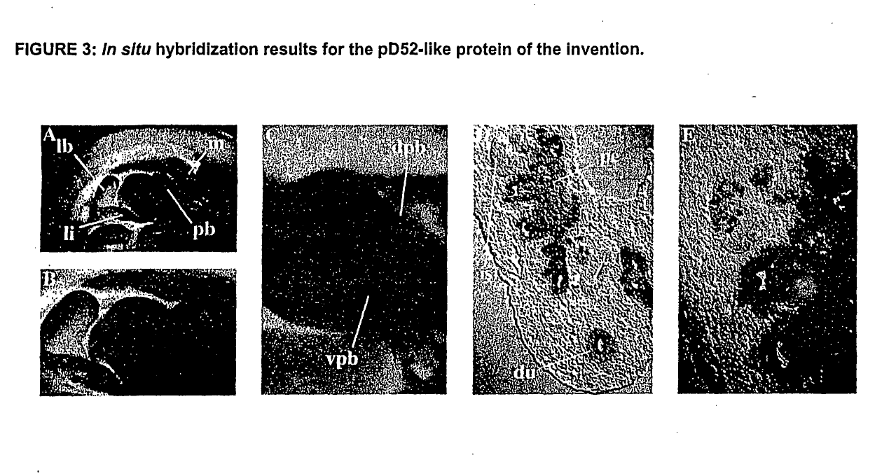

In situ hybridization experiments using the D52 proteins described in this invention were done on whole mounts of 5-day-old chick embryos (FIGS. 3, a, b, and c) and on sectioned pancreatic bud tissue (FIG. 3d, e). The hybridizations show that D52 transcripts of the invention are exclusively expressed in the embryonic pancreatic epithelium (pe, FIG. 3) and not in the surrounding mesenchyme.

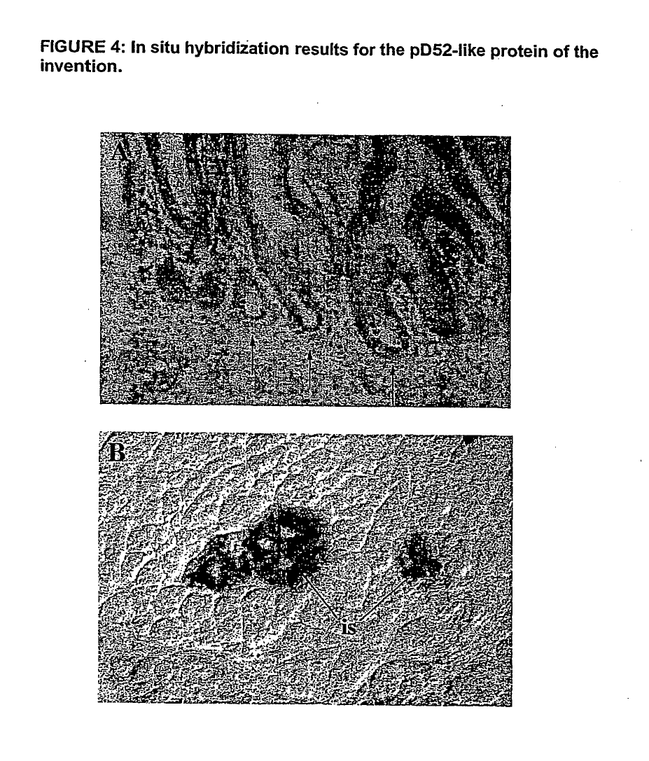

Hybridization experiments using human duodenum (FIG. 4a) and human pancreas (FIG. 4b) show a specific expression of the protein of the invention in crypt cells of the intestine (FIG. 4a) and in pancreatic islet cells (FIG. 4b).

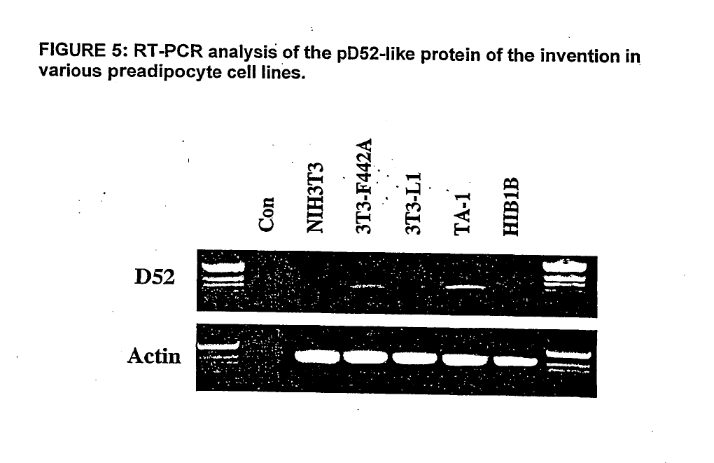

FIG. 5 shows the RT-PCR analysis of D52 expression in various preadipocyte cell lines. D52 is expressed in all preadipocte cell lines tested but is absent in simple fibroblast cells (NIH3T3). The lower panel in FIG. 6 depicts actin to control for the integrity of the cDNA samples.

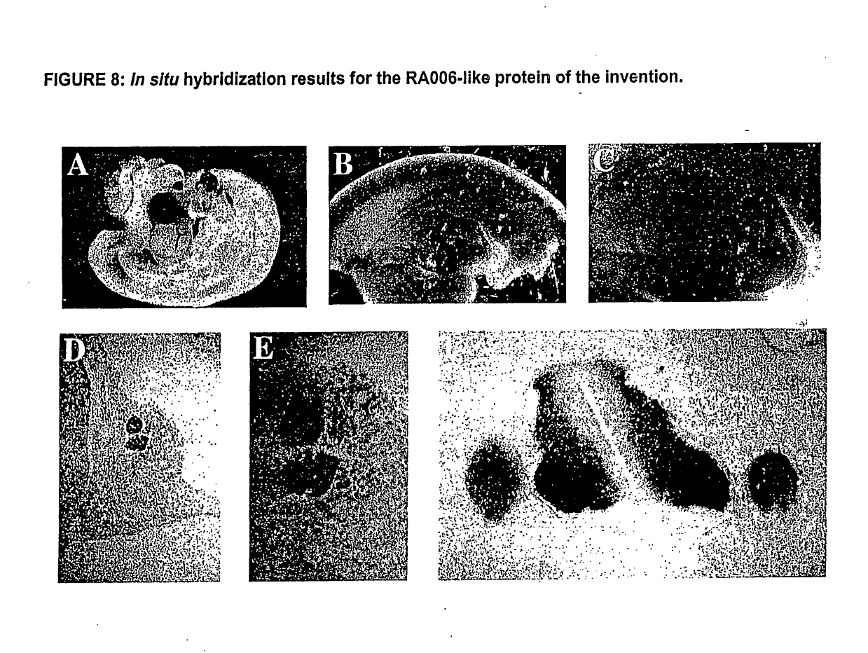

In situ hybridization experiments using the RA006 protein described in this invention were done on whole mounts of 4 (FIG. 8A) and 5-day-old chick embryos (FIGS. 8B and C) and on sectioned pancreatic bud tissue (FIG. 8D and E) and sections of neural tissue (FIG. 8F). The hybridizations show that transcripts of the invention are exclusively expressed in a subset of islet cells of the pancreas (FIG. 8D and E).

Hybridizations carried out on crossections of the neural tube and dorsal root ganglia (FIG. 8F) show a specific expression of RA006 transcripts of the invention in lateral areas of the neural tube as well as the dorsal root ganglia.

The invention also encompasses pD52 or pRA006 variants. A preferred pD52 or pRA006 variant is one having at least 80%, and more preferably 90%, amino acid sequence similarity to the pD52 or pRA006 amino acid sequence (SEQ ID NO:2 or SEQ ID NO:4, respectively). A most preferred pD52 or pRA006 variant is one having at least 95% amino acid sequence similarity to SEQ ID NO:1 or SEQ ID NO:3, respectively.

The invention also encompasses polynucleotides which encode pD52 or pRA006. Accordingly, any nucleic acid sequence which encodes the amino acid sequence of pD52 or pRA006 can be used to generate recombinant molecules which express pD52 or pRA006. In a particular embodiment, the invention encompasses the polynucleotide comprising the nucleic acid sequence of SEQ ID NO:1 as shown in FIG. 1A. or SEQ ID NO:3 as shown in FIG. 6A. It will be appreciated by those skilled in the art that as a result of the degeneracy of the genetic code, a multitude of nucleotide sequences encoding pD52 or pRA006, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequence of naturally occurring pD52 or pRA006, and all such variations are to be considered as being specifically disclosed. Although nucleotide sequences which encode pD52 or pRA006 and its variants are preferably capable of hybridizing to the nucleotide sequence of the naturally occurring pD52 under appropriately selected conditions of stringency, it may be advantageous to produce nucleotide sequences encoding pD52 or pRA006 or its derivatives possessing a substantially different codon usage. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding pD52 or pRA006 and its derivatives without altering the encoded amino acid sequences include the production of RNA transcripts having more desirable properties, such as a greater half-life, than transcripts produced from the naturally occurring sequence. The invention also encompasses production of DNA sequences, or portions therof, which encode pD52 or pRA006 and its derivatives, entirely by synthetic chemistry. After production, the synthetic sequence may be inserted into any of the many available expression vectors and cell systems using reagents that are well known in the art. Moreover, synthetic chemistry may be used to introduce mutations into a sequence encoding pD52 or pRA006 or any portion thereof.

Also encompassed by the invention are polynucleotide sequences that are capable of hybridizing to the claimed nucleotide sequences, and in particular, those shown in SEQ ID NO:2 or SEQ ID NO:4 or sequences complementary thereto, under various conditions of stringency. Hybridization conditions are based on the melting temperature (Tm) of the nucleic acid binding complex or probe, as taught in Wahl, G. M. and S. L. Berger (1987, Methods Enzymol. 152:399-407) and Kimmel, A. R. (1987, Methods Enzymol. 152:507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed.

Altered nucleic acid sequences encoding pD52 or pRA006 which are encompassed by the invention include deletions, insertions, or substitutions of different nucleotides resulting in a polynucleotide that encodes the same or a functionally equivalent pD52 or pRA006. The encoded protein may also contain deletions, insertions, or substitutions of amino acid residues which produce a silent change and result in a functionally equivalent pD52 or pRA006. Deliberate amino acid substitutions may be made on the basis of similarity in polarity, charge, solubility, hydrophobicity, hydrophilicity, and/or the amphipathic nature of the residues as long as the biological activity of pD52 or pRA006 is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; phenylalanine and tyrosine.

Also included within the scope of the present invention are alleles of the genes encoding pD52 or pRA006. As used herein, an “allele” or “allelic sequence” is an alternative form of the gene which may result from at least one mutation in the nucleic acid sequence. Alleles may result in altered mRNAs or polypeptides whose structures or function may or may not be altered. Common mutational changes which give rise to alleles are generally ascribed to natural deletions, additions, or substitutions of nucleotides. Each of these types of changes may occur alone, or in combination with the others, one or more times in a given sequence.

Methods for DNA sequencing which are well known and generally available in the art may be used to practice any embodiments of the invention. The nucleic acid sequences encoding pD52 or pRA006 may be extended utilizing a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). Inverse PCR may also be used to amplify or extend sequences using divergent primers based on a known region (Triglia, T. et al. (1988) Nucleic Acids Res. 16:8186). The primers may be designed using OLIGO 4.06 primer analysis software (National Biosciences Inc., Plymouth, Minn.), or another appropriate program, preferably to have 22-30 nucleotides in length, to have a GC content of 50% or more, and to anneal to the target sequence at temperatures about 68° C.-72° C.

Another method which may be used is capture PCR which involves PCR amplification of DNA fragments adjacent to a known sequence in human and yeast artificial chromosome DNA (Lagerstrom, M. et al. (PCR Methods Applic. 1:111-119). Another method which may be used to retrieve unknown sequences is that of Parker, J. D. et al. (1991; Nucleic Acids Res. 19:3055-3060). Additionally, one may use PCR, nested primers, and PROMOTERFINDER libraries to walk in genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences which contain the 5′ regions of genes. Use of a randomly primed library may be especially preferable for situations in which an oligo d(T) library does not yield a full-length cDNA. Genomic libraries may be useful for extension of sequence into the 5′ and 3′ non-transcribed regulatory regions. Capillary electrophoresis systems which are commercially available may be used to analyze the size or confirm the nucleotide sequence of sequencing or PCR products.

In another embodiment of the invention, polynucleotide sequences or fragments thereof which encode pD52 or pRA006, or fusion proteins or functional equivalents thereof, may be used in recombinant DNA molecules to direct expression of pD52 or pRA006 in appropriate host cells. Due to the inherent degeneracy of the genetic code, other DNA sequences which encode substantially the same or a functionally equivalent amino acid sequence may be produced and these sequences may be used to clone and express pD52 or pRA006. As will be understood by those of skill in the art, it may be advantageous to produce pD52 or pRA006-encoding nucleotide sequences possessing non-naturally occurring codons. For example, codons preferred by a particular prokaryotic or eukaryotic

host can be selected to increase the rate of protein expression or to produce a recombinant RNA transcript having desirable properties, such as a half-life which is longer than that of a transcript generated from the naturally occurring sequence. The nucleotide sequences of the present invention can be engineered using methods generally known in the art in order to alter pD52 or pRA006 encoding sequences for a variety of reasons, including but not limited to, alterations, which modify the cloning, processing, and/or expression of the-gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences encoding pD52 or pRA006 may be ligated to a heterologous sequence to encode a fusion protein. For example, to screen peptide libraries for inhibitors of pD52 or pRA006 activity, it may be useful to encode a chimeric pD52 or pRA006 protein that can be recognized by a commercially available antibody. A fusion protein may also be engineered to contain a cleavage site located between the pD52 or pRA006 encoding sequence and the heterologous protein sequence, so that pD52 or pRA006 may be cleaved and purified away from the heterologous moiety. In another embodiment, sequences encoding pD52 or pRA006 may be synthesized, in whole or in part, using chemical methods well known in the art (see Caruthers, M. H. et al. (1980) Nucl. Acids Res. Symp. Ser. 7:215-223, Horn, T. et al. (1980) Nucl. Acids Res. Symp. Ser. 7:225-232). Alternatively, the protein itself or a portion thereof may be produced using chemical methods. For example, peptide synthesis can be performed using various solid-phase techniques (Roberge, J. Y. et al. (1995) Science 269:202-204) and automated synthesis may be achieved, for example, using the ABI 431A peptide synthesizer (Perkin Elmer). Additionally, the amino acid sequence of pD52 or pRA006, or any part thereof, may be altered during direct synthesis and/or combined using chemical methods with sequences from other proteins, or any part thereof, to produce a variant polypeptide. In order to express a biologically active pD52 or pRA006, the nucleotide sequences encoding pD52 or pRA006 or functional equivalents, may be inserted into appropriate expression vector, i.e. a vector which contains the necessary elements for the transcription and translation of the inserted coding sequence. Methods which are well known to those skilled in the art may be used to construct expression vectors containing sequences encoding pD52 or pRA006 and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

A variety of expression vector/host systems may be utilized to contain and express sequences encoding pD52 or pRA006. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g. baculovirus); plant cell systems transformed with virus expression vectors (e.g. cauliflower mosaic virus, CaMV; tobacco mosaic virus, TMV) or with bacterial expression vectors (e.g. Ti or PBR322 plasmids); or animal cell systems. The “control elements” or “regulatory sequences” are those non-translated regions of the vector-enhancers, promoters, 5′ and 3′ untranslated regions—which interact with host cellular proteins to carry out transcription and translation. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT phagemid (Stratagene, LaJolla, Calif.) or PSPORT1 plasmid (Gibco BRL) and the like may be used. The baculovirus polyhedrin promoter may be used in insect cells. Promoters and enhancers derived from the genomes of plant cells (e.g. heat shock, RUBISCO; and storage protein genes) or from plant viruses (e.g. viral promoters and leader sequences) may be cloned into the vector. In mammalian cell systems, promoters from mammalian genes or from mammalian viruses are preferable. If it is necessary to generate a cell line that contains multiple copies of the sequence encoding pD52 or pRA006, vectors based on SV40 or EBV may be used with an appropriate selectable marker.

In bacterial systems, a number of expression vectors may be selected depending upon the use intended for pD52 or pRA006. For example, when large quantities of pD52 or pRA006 are needed for the induction of antibodies, vectors which direct high level expression of fusion proteins that are readily purified may be used. Such vectors include, but are not limited to, the multifunctional E. coli cloning and expression vectors such as the BLUESCRIPT phagemid (Stratagene), PIN vectors (Van Heeke and Schuster (1989), J. Biol. Chem. 264:5503-5509) or the PGEX vectors (Promega, Madison, Wis.). In the yeast, Saccharomyces cerevisiae, a number of vectors containing constitutive or inducible promoters such as alpha factor, alcohol oxidase, and PGH may be used. For reviews, see Ausubel et al., (supra) and Grant et al. (1987) Methods Enzymol. 153:516-544.

In cases where plant expression vectors are used, the expression of sequences encoding pD52 or pRA006 may be driven, e.g. by viral promoters such as the 35S and 19S promoters of CaMV alone or in combination with the omega leader sequence from TMV (Takamatsu, N. (1987)

EMBO J. 6:307-311), or plant promoters such as the small subunit of RUBISCO or heat shock promoters (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196.

An insect system may also be used to express pD52 or pRA006. For example, in one such system, Autographa californica nuclear polyhedrosis virus (AcNPV) is used as a vector to express foreign genes in Spodoptera frugiperda cells or in Trichoplusia larvae. The sequences encoding pD52 or pRA006 may be cloned into a non-essential region of the virus, such as the polyhedrin gene, and place under control of the polyhedrin promoter. Successful insertion of pD52 or pRA006 will render the polyhedrin gene inactive and produce recombinant virus lacking coat protein. The recombinant viruses may then be used to infect, for example, S. frugiperda cells of Trichoplusia larvae in which pD52 or pRA006 may be expressed (Engelhard, E. K. et al. (1994) Proc. Nat.

Acad. Sci. 91:3224-3227).

In mammalian host cells, a number of viral-based expression systems may be utilized. In cases where an adenovirus is used as an expression vector, sequences encoding pD52 or pRA006 may be ligated into an adenovirus transcription/translation complex consisting of the late promoter and tripartite leader sequence. Insertion in a non-essential E1 or E3 region of the viral genome may be used to obtain a viable virus which is capable of expressing pD52 or pRA006 in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding pD52 or pRA006. Such signals include the ATG initiation codon and adjacent sequences. If desired, exogenous translational control signals including the ATG initiation codon may be provided. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probi. Cell Differ. 20:125-162).

For long-term, high-yield production of recombinant proteins, stable expression is preferred. For example, cell lines which stably express pD52 or pRA006 may be transformed using expression vectors which may contain viral origins of replication and/or endogenous expression elements and a selectable marker gene on the same or on a separate vector. Following the introduction of the vector, a selection for stably transformed cells is carried out. Clones of stably transformed cells may be proliferated using tissue culture techniques appropriate to the cell type. Any number of selection systems may be used to recover transformed cell lines, e.g. the herpes simplex virus thymidine kinase (Wigler, M. et al. (1977) Cell 11:223-32) and adenine phosphoribosyltransferase (Lowy, I. et al. (1980) Cell 22:817-23) genes which can be employed in tk- or aprt-cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150:1-14) and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). Recently, the use of visible markers has gained popularity with such markers as anthocyanins, β glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

The presence and/or expression of that the gene of interest may be confirmed by appropriate methods. These include, but are not limited to, DNA-DNA, or DNA-RNA hybridizations and protein bioassay or immuno-assay techniques which include membrane, solution, or chip based technologies for the detection and/or quantification of nucleic acid or protein.

The presence of polynucleotide sequences encoding pD52 or pRA006 can be detected by DNA-DNA or DNA-RNA hybridization or amplification using probes or portions or fragments of polynucleotides encoding pD52 or pRA006. Nucleic acid amplification based assays involve the use of oligonucleotides or oligomers based on the sequences encoding pD52 or pRA006 to detect transformants containing DNA or RNA encoding pD52 or pRA006. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

A variety of protocols for detecting and measuring the expression of pD52 or pRA006, using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on pD52 or pRA006 is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding pD52 or pRA006 include oligolabeling, nick translation, end-labeling or PCR amplification using a labeled nucleotide.

Alternatively, the sequences encoding pD52 or pRA006, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits (Pharmacia & Upjohn, (Kalamazoo, Mich.); Promega (Madison Wis.); and U.S. Biochemical Corp., (Cleveland, Ohio).

Suitable reporter molecules or labels, which may be used, include radio-nuclides, enzymes, fluorescent, chemiluminescent, or chromogenic agents as well as substrates, cofactors, inhibitors, magnetic particles, and the like.

Host cells transformed with nucleotide sequences encoding pD52 or pRA006 may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides which encode pD52 or pRA006 may be designed to contain signal sequences which direct secretion of pD52 or pRA006 through a prokaryotic or eukaryotic cell membrane. Other recombinant constructions may be used to join sequences encoding pD52 or pRA006 to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAG extension/affinity purification system (Immunex Corp., Seattle, Wash.) The inclusion of cleavable linker sequences such as those specific for Factor XA or enterokinase (Invitrogen, San Diego, Calif.) between the purification domain and pD52 or pRA006 may be used to facilitate purification. One such expression vector provides for expression of a fusion protein containing pD52 or pRA006 and a nucleic acid encoding β histidine residues preceding a thioredoxin or an enterokinase cleavage site. The histidine residues facilitate purification on IMIAC (immobilized metal ion affinity chromotagraphy as described in Porath, J. et al. (1992, Prot. Exp. Purif. 3: 263-281) while the enterokinase cleavage site provides a means for purifying pD52 or pRA006 from the fusion protein. A discussion of vectors which contain fusion proteins is provided in Kroll, D. J. et al. (1993; DNA Cell Biol. 12:441-453). In addition to recombinant production, fragments of pD52 or pRA006 may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A peptide synthesizer (Perkin Elmer). Various fragments of pD52 or pRA006 may be chemically synthesized separately and combined using chemical methods to produce the full length molecule.

Diagnostics and Therapeutics

The proteins of this invention are specifically expressed in pancreatic cells such as islet cells and pancreatic stem cells presumed to reside in the pancreatic epithelium. pD52 is further expressed in adult stem cells residing in the intestinal crypts of the duodenum, and is also involved in the differentiation of fibroblasts into preadipocytic cells. RA006 is further expressed in the eye as well as the central and peripheral nervous system. Therefore, the nucleic acid and proteins of the invention are useful as markers for identifying specific cell types. Further, they are useful in diagnostic and therapeutic applications implicated, for example but not limited to, in metabolic disorders and dysfunctions like diabetes and obesity, and neurodegenrative diseses, and other diseases and disorders, particularly those associated with the above cell types. Hence the proteins of the invention are useful as a diagnostic marker and/or as a target for effector molecules, e.g. small molecules, in treatment of diabetes and/or obesity and other metabolic disorders and other diseases.

Preferred therapeutic uses for the invention(s) are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues.

The nucleic acids and proteins of the invention are useful in therapeutic applications implicated in various diseases and disorders. For example, but not limited to, a cDNA encoding the pD52- or pRA006 -like protein of the invention may be useful in gene therapy, and the pD52- or pRA006 -like protein of the invention may be useful when administered to a subject in need thereof. By way of non-limiting example, the compositions of the present invention will have efficacy for treatment of patients suffering from,.for example, but not limited to, in metabolic disorders like obesity and diabetes, and other diseases and disorders. The novel nucleic acid encoding the pD52-like or pRA006-like protein of the invention, or fragments thereof, may further be useful in diagnostic applications, wherein the presence or amount of the nucleic acid or the protein are to be assessed.

Further, antibodies which bind immunospecifically to the novel substances of the invention may be used in therapeutic or diagnostic methods. For example, in one aspect, antibodies which are specific for pD52 or pRA006 may be used directly as an antagonist, or indirectly as a targeting or delivery mechanism for bringing a pharmaceutical agent to cells or tissue which express pD52 or pRA006. The antibodies may be generated using methods that are well known in the art. Such antibodies may include, but are not limited to, polyclonal, monoclonal, chimeric, single chain, Fab fragments, and fragments produced by a Fab expression library. Neutralizing antibodies, (i.e. those which inhibit dimer formation) are especially preferred for therapeutic use.

For the production of antibodies, various hosts including goats, rabbits, rats, mice, humans, and others, may be immunized by injection with pD52 or pRA006 or any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in human, BCG (Bacille Calmette-Guerin) and Corynebacterium parvum are especially preferable. It is preferred that the peptides, fragments, or oligopeptides used to induce antibodies to pD52 or pRA006 have an amino acid sequence consisting of at least five amino acids, and more preferably at least 10 amino acids. Short stretches of pD52 or pRA006 amino acids may be fused with those of another protein such as keyhole limpet hemocyanin and anti-body produced against the chimeric molecule.

Monoclonal antibodies to pD52 or pRA006 may be prepared using any technique which provides for the production of antibody molecules by continuous cell lines in culture. These include, but are not limited to, the hybridoma technique, the human B-cell hybridoma technique, and the EBV-hybridoma technique (Köhler, G. et al. (1975) Nature 256:495-497; Kozbor, D. et al. (1985) J. Immunol. Methods 81:31-42; Cote, R. J. et al. (Proc. Natl. Acad. Sci. 80:2026-2030; Cole, S. P. et al. (1984) Mol. Cell Biol. 62:109-120). In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce pD52 or pRA006-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. 88:11120-3). Anti-bodies may also be producing by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Antibody fragments which contain specific binding sites for pD52 or pRA006 may also be generated. For example, such fragments include, but are not limited to, the F(ab′)₂ fragments which can be produced by pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse, W. D. et al. (1989) Science 254:1275-1281).

In another embodiment of the invention, polynucleotides encoding pD52 or pRA006, or any fragment thereof, or antisense molecules, may be used for therapeutic purposes. In one aspect, antisense molecules to the polynucleotide encoding pD52 or pRA006 may be used in situations in which it is desirable to block the transcription of the mRNA. In particular, cells may be transformed with sequences complementary to polynucleotides encoding pD52 or pRA006. Thus, antisense molecules may be used to modulate pD52 or pRA006 activity, or to achieve regulation of gene function. Such technology is now well know in the art, and sense or antisense oligomers or larger fragments, can be designed from various locations along the coding or control regions of sequences encoding pD52 or pRA006. Expression vectors derived from retroviruses, adenovirus, herpes or vaccinia viruses, or from various bacterial plasmids may be used for delivery of nucleotide sequences to the targeted organ, tissue or cell population. Methods which are well known to those skilled in the art can be used to construct recombinant vectors which will express anti-sense molecules complementary to the polynucleotides of the gene encoding pD52 or pRA006. These techniques are described both in Sambrook et al. (supra) and in Ausubel et al. (supra). Genes encoding pD52 or pRA006 can be turned off by transforming a cell or tissue with expression vectors which express high levels of a polynucleotide or fragment thereof which encodes pD52 or pRA006. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, DNA, RNA, or PNA, to the control regions of the gene encoding pD52 or pRA006, i.e., the promoters, enhancers, and introns. Oligonucleotides derived from the transcription initiation site, e.g. between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors, or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples which may be used include engineered hammerhead motif ribozyme molecules that can be specifically and efficiently catalyze endonucleolytic cleavage of sequences encoding pD52 or pRA006. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Antisense molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding pD52 or pRA006. Such DNA sequences may be incorporated into a variety of vectors with suitable RNA polymerase promoters such as T7 or SP6. Alternatively, these cDNA constructs that synthesize antisense RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. Further, the nucleic acid backbone may be modified, e.g. substituted by a peptide backbone, such as in the production of PNAs. Furthermore, nontraditional bases such as inosine, queosine, and wybutosine, as well as acetyl-, methyl-, thio- and similarly modified forms of adenine, cytidine, guanine, thymine, and/or uridine may be used, which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may comprise an active ingredient selected from D52 or RA006 nucleic acids, pD52 or pRA006, antibodies to pD52 or pRA006, mimetics, agonists, antagonists, or inhibitors of pD52 or pRA006. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones. The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.). Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypro-pylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate. Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coating for product identification or to characterize the quantity of active compound, i.e. dosage. Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxy-methyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g. by means of conventional mixing, dissolving, granulating, dragee-making, levigating, emulsifying, encapsulating, entrapping, or lyophilizing processes. The pharmaceutical composition may be provided as a salt and can be formed with many acids, including but not limited to, hydrochloric, sulfuric, acetic, lactic, tartaric, malic, succinic, etc. In other cases, the preferred preparation may be a lyophilized powder which may contain any or all of the following: 1-50 mM histidine, 0.1%-2% sucrose, and 2-7% mannitol, at a pH range of 4.5 to 5.5, that is combined with buffer prior to use. After pharmaceutical compositions have been prepared, they can be placed in an appropriate container and labeled for treatment of an indicated condition. For administration of pD52 or pRA006, such labeling would include, amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective does can be estimated initially either in cell culture assays, e.g. of preadipoctic cell lines, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example pD52 or pRA006 or fragments thereof, antibodies of pD52 or pRA006, which is effective for the treatment of a specific condition. Therapeutic efficacy can toxicity may be determined by standard pharmaceutical procedures in cell cultures or experimental animals, e.g. ED50 (the does therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, dependirig upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind pD52 or pRA006 may be used for the diagnosis of conditions or diseases characterized by expression of pD52 or pRA006, or in assays to monitor patients being treated with pD52 or pRA006, agonists, antagonists or inhibitors. The antibodies useful for diagnostic purposes may be prepared in the same manner as those described above for therapeutics. Diagnostic assays for pD52 or pRA006 include methods which utilize the anti-body and a label to detect pD52 or pRA006 in human body fluids or extracts of cells or tissues. The antibodies may be used with or without modification, and may be labeled by joining them, either covalently or non-covalently, with a reporter molecule. A wide variety of reporter molecules which are known in the art may be used several of which are described above.

A variety of protocols including ELISA, RIA, and FACS for measuring pD52 or pRA006 are known in the art and provide a basis for diagnosing altered or abnormal levels of pD52 or pRA006 expression. Normal or standard values for pD52 or pRA006 expression are established by combining body fluids or cell extracts taken from normal mammalian subjects, preferably human, with antibody to pD52 or pRA006 under conditions suitable for complex formation. The amount of standard complex formation may be quantified by various methods, but preferably by photometric, means. Quantities of pD52 or pRA006 expressed in control and disease, samples from biopsied tissues are compared with the standard values. Deviation between standard and subject values establishes the parameters for diagnosing disease.

In another embodiment of the invention, the polynucleotides encoding pD52 or pRA006 may be used for diagnostic purposes. The polynucleotides which may be used include oligonucleotide sequences, antisense RNA and DNA molecules, and PNAs. The polynucleotides may be used to detect and quantitate gene expression in biopsied tissues in which expression of pD52 or pRA006 may be correlated with disease. The diagnostic assay may be used to distinguish between absence, presence, and excess expression of pD52 or pRA006, and to monitor regulation of pD52 or pRA006 levels during therapeutic intervention.

In one aspect, hybridization with PCR probes which are capable of detecting polynucleotide sequences, including genomic sequences, encoding pD52 or pRA006 or closely related molecules, may be used to identify nucleic acid sequences which encode pD52 or pRA006. The specificity of the probe, whether it is made from a highly specific region, or a less specific region, and the stringency of the hybridization or amplification (maximal, high, intermediate, or low) will determine whether the probe identifies only naturally occurring sequences encoding pD52 or pRA006, alleles, or related sequences. Probes may also be used for the detection of related sequences, and should preferably contain at least 50% of the nucleotides from any of the pD52 or pRA006 encoding sequences. The hybridization probes of the subject invention may be DNA or RNA and derived from the nucleotide sequence of SEQ ID NO:1 or SEQ ID NO:3 or from genomic sequence including promoter, enhancer elements, and introns of the naturally occurring D52 or RA006 genes. Means for producing specific hybridization probes for DNAs encoding pD52 or pRA006 include the cloning of nucleic acid sequences encoding pD52 or pRA006 or pD52 or pRA006 derivatives into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as ³²p or ³⁵S, or enzymatic labels, such as alkaline phosphatase coupled to the probe via avidin/biotin coupling systems, and the like.

Polynucleotide sequences specific for D52 or RA006 may be used for the diagnosis of conditions or diseases which are associated with expression of pD52 or pRA006. Examples of such conditions or diseases include, but are not limited to, pancreatic diseases and disorders, including diabetes. Polynucleotide sequences specific for D52 or RA006 may also be used to monitor the progress of patients receiving treatment for pancreatic diseases and disorders, including diabetes. The polynucleotide sequences may be used in Southern or northern analysis, dot blot, or other membrane-based technologies; in PCR technologies; or in dip stick, pin, ELISA or chip assays utilizing fluids or tissues from patient biopsies to detect altered pD52 or pRA006 expression. Such qualitative or quantitative methods are well known in the art.

In a particular aspect, the nucleotide sequences specific for D52 or RA006 may be useful in assays that detect activation or induction of various pancreatic diseases and disorders, including diabetes, particularly those mentioned above. The nucleotide sequences may be labeled by standard methods, and added to a fluid or tissue sample from a patient under conditions suitable for the formation of hybridization complexes. After a suitable incubation period, the sample is washed and the signal is quantitated and compared with a standard value. The presence of altered levels of nucleotide sequences encoding pD52 or pRA006 in the sample indicates the presence and/or the severity of an associated disease. Such assays may also be used to evaluate the efficacy of a particular therapeutic treatment regimen in animal studies, in clinical trials, or in monitoring the treatment of an individual patient.

In order to provide a basis for the diagnosis of disease associated with expression of pD52 or pRA006, a normal or standard profile for expression is established. This may be accomplished by combining body fluids or cell extracts taken from normal subjects, either animal or human, with a sequence, or a fragment thereof, which encodes pD52 or pRA006, under conditions suitable for hybridization or amplification. Standard hybridization may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridization assays may be repeated on a regular basis to evaluate whether the level of expression in the patient begins to approximate that which is observed in the normal patient. The results obtained from successive assays may be used to show the efficacy of treatment over a period ranging from several days to months.

With respect to pancreatic diseases and disorders, including diabetes, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the pancreatic diseases and disorders. Additional diagnostic uses for oligonucleotides designed from the sequences encoding pD52 or pRA006 may involve the use of PCR. Such oligomers may be chemically synthesized, generated enzymatically, or produced from a recombinant source. Oligomers will preferably consist of two nucleotide sequences, one with sense orientation (5′.fwdarw.3′) and another with antisense (3′.rarw.5′), employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Methods which may also be used to quantitate the expression of pD52 or pRA006 include radiolabeling or biotinylating nucleotides, coamplification of a control nucleic acid, and standard curves onto which the experimental results are interpolated (Melby, P. C. et al. (1993) J. Immunol. Methods, 159:235-244; Duplaa, C. et al. (1993) Anal. Biochem. 212:229-236. The speed of quantitation of multiple samples may be accelerated by running the assay in an ELISA format where the oligomer of interest is presented in various dilutions and a spectrophotometric or colorimetric response gives rapid quantitation.

In another embodiment of the invention, the nucleic acid sequences which are specific for D52 or RA006 may also be used to generate hybridization probes which are useful for mapping the naturally occurring genomic sequence. The sequences may be mapped to a particular chromosome or to a specific region of the chromosome using well known techniques. Such techniques include FISH, FACS, or artificial chromosome constructions, such as yeast artificial chromosomes, bacterial artificial chromosomes, bacterial P1 constructions or single chromosomencDNA libraries as reviewed in Price, C. M. (1993) Blood Rev. 7:127-134, and Trask, B. J. (1991) Trends Genet. 7:149-154. FISH (as described in Verma et al. (1988) Human Chromosomes: A Manual of Basic Techniques, Pergamon Press, New York, N.Y.) may be correlated with other physical chromosome mapping techniques and genetic map data. Examples of genetic map data can be found in the 1994 Genome Issue of Science (265:1981f). Correlation between the location of the gene encoding pD52 or pRA006 on a physical chromosomal map and a specific disease, or predisposition to a specific disease, may help delimit the region of DNA associated with that genetic disease.

The nucleotide sequences of the subject invention may be used to detect differences in gene sequences between normal, carrier, or affected individuals. In situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11 q22-23 (Gatti, R. A. et al. (1988) Nature 336:577-580), any sequences mapping to that area may represent associated or regulatory genes for further investigation. The nucleotide sequence of the subject invention may also be used to detect differences in the chromosomal location due to translocation, inversion, etc. among normal, carrier, or affected individuals.

In another embodiment of the invention, pD52 or pRA006, its catalytic or immunogenic fragments or oligopeptides thereof, can be used for screening libraries of compounds in any of a variety of drug screening techniques. The fragment employed in such screening may be free in solution, affixed to a solid support, borne on a cell surface, or located intra-cellularly. The formation of binding complexes, between pD52 or pRA006 and the agent tested, may be measured.

Another technique for drug screening which may be used provides for high throughput screening of compounds having suitable binding affinity to the protein of interest as described in published PCT application WO84/03564. In this method, as applied to pD52 or pRA006 large numbers of different small test compounds, e.g. peptides or low-molecular weight organic compounds, are provided or synthesized on a solid substrate, such as plastic pins or some other surface. The test compounds are reacted with pD52 or pRA006, or fragments thereof, and washed. Bound pD52 or pRA006 is then detected by methods well known in the art. Purified pD52 or pRA006 can also be coated directly onto plates for use in the aforementioned drug screening techniques. Alternatively, non-neutralizing antibodies can be used to capture the peptide and immobilize it on a solid support. In another embodiment, one may use competitive drug screening assays in which neutralizing antibodies capable of binding pD52 or pRA006 specifically compete with a test compound for binding pD52 or pRA006. In this manner, the antibodies can be used to detect the presence of any peptide which shares one or more antigenic determinants with pD52 or pRA006. In additional embodiments, the nucleotide sequences which encode pD52 or pRA006 may be used in any molecular biology techniques that have yet to be developed, provided the new techniques rely on properties of nucleotide that are currently known, including, but not limited to, such properties as the triplet genetic code and specific base pair interactions.

The examples below are provided to illustrate the subject invention and are not included for the purpose of limiting the invention.

### EXAMPLES

I. DPd6 Chick cDNA Library Construction

The Chick DPd6 cDNA library was constructed from dorsal pancreatic buds dissected from 6 day old chick embryos. The frozen tissue was homogenized and lysed using a Brinkmann POLYTRON homogenizer PT-3000 (Brinkman Instruments, Westbury, N.J.) in guanidinium isothiocyanate solution. The lysates were centrifuged over a 5.7 M CsCl cushion using as Beckman SW28 rotor in a Beckman L8-70M ultracentrifuge (Beckman Instruments, Fullerton, Calif.) for 18 hours at 25,000 rpm at ambient temperature. The RNA was extracted with acid phenol pH 4.7, precipitated using 0.3 M sodium acetate and 2.5 volumes of ethanol, resuspended in RNase-free water, and DNase treated at 37° C. The RNA extraction was repeated with acid phenol pH 4.7 and precipitated with sodium acetate and ethanol as before. The mRNA was then isolated using the Micro-FastTrack 2.0 mRNA isolation kit (Invitrogen, Groningen, Netherlands) and used to construct the cDNA libraries. The mRNAs were handled according to the recommended protocols in the SUPERSCRIPT cDNA synthesis and plasmid cloning system (Cat. #18248-013. Gibco/BRL). Following transformation into DH10B host cells, single colonies were picked and the subjected to PCR in order to amplify the cloned cDNA insert. Amplified PCR fragments representing single cDNA inserts were subsequently in vitro transcribed to generate Digoxygenin labelled RNA probes (Roche). The RNA probes were used in a whole-mount in situ screen to determine the expression of their respective gene products in early chick embryos. The plasmids pRA290 and pRA006 containing the chicken D52-like or RA006-like gene were identified because of their striking expression in the pancreatic epithelium.

II. In Situ Hybridizations and RT-PCR Analysis

Whole-mount in situ hybridizations were performed according to standard protocols as known to those skilled in the art and as described previously (for example, Pelton, R. W. et al., (1990) Development 110,609-620; Belo, J. A. et al., (1997) Mech. Dev. 68, 45-57)(see FIGS. 3, 4, and 8). Isolation of total RNA from cell culture was carried out using trizol (Life Technologies, Karlsruhe, Germany). Reverse-transcriptase-polymerase chain reaction protocols have been described previously (Dohrmann, C. E. et al. (1996) Dev. Biol. 175, 108-117)(see FIG. 5).

Ill. Isolation and Sequencing of cDNA Clones

Plasmid DNA was released from the cells and purified using the REAL PREP 96-well plasmid isolation kit (Catalog #26173, QIAGEN). This kit enabled the simultaneous purification of 96 samples in a 96-well block using multi-channel reagent dispensers. The protocol recommended by the manufacturer was employed except for the following changes, as indicated below: (i) the bacteria were cultured in 1 ml of sterile Terrific Broth (Catalog #22711, LIFE TECHNOLOGIESTM, Gaithersburg, Md., USA) with carbenicillin at 25 mg/L and glycerol at 0.4%; (ii) after inoculation, the cultures were incubated for 19 hours and at the end of incubation, the cells were lysed with 0.3 ml of lysis buffer; and (iii) following isopropanol precipitation, the plasmid DNA pellet was resuspended in 0.1 ml of distilled water. After the last step in the protocol, samples were transferred to a 96-well block for storage at 4° C. The cDNAs were sequenced by GATC Biotech AG (Konstanz, Germany) accoding to standard protocols known to those skilled in the art.

IV. Homology Searching of cDNA Clones and Their Deduced Proteins

After the reading frame was determined, the nucleotide sequences of the invention as well as the amino acid sequences deduced from them were used as query sequences against databases such as GenBank, Swiss-Prot, BLOCKS, and Pima II. These databases, which contain previously identified and annotated sequences, were searched for regions of homology (similarity) using BLAST, which stands for Basic Local Alignment Search Tool (Altschul S. F. (1993) J. Mol. Evol. 36:290-300; Altschul, S. F. et al. (1990) J. Mol. Biol. 215:403-10). BLAST produced alignments of both nucleotide and amino acid sequences to determine sequence similarity. Because of the local nature of the alignments, BLAST was especially useful in determining exact matches or in identifying homologs which may be of prokaryotic (bacterial) or eukaryotic (animal, fungal, or plant) origin. Other algorithms such as the one described in Smith et al. (1992, Protein Engineering 5:35-51), incorporated herein by reference, could have been used when dealing with primary sequence patterns and secondary structure gap penalties. The BLAST approach, as detailed in Karlin et al. (supra) and incorporated herein by reference, searched for-matches between a query sequence and a database sequence. BLAST evaluated the statistical significance of any matches found, and reported only those matches that satisfy the user-selected threshold of significance. In this application, threshold was set at 10-25 for nucleotides and 10-14 for peptides. Nucleotide sequences were searched against the GenBank databases for primate, rodent, and other mammalian sequences; and deduced amino acid sequences from the same clones were then searched against GenBank functional protein databases, mammalian, vertebrate, and eukaryote for homology.

V. Extension of pD52 or pRA006 Encoding Polynucleotides to Full Length or Recovery of Regulatory Sequences

Full length pD52-encoding or pRA006-encoding nucleic acid sequence (SEQ ID NO:1 or SEQ ID NO:3, respectively) is used to design oligonucleotide primers for extending a partial nucleotide sequence to full length or for obtaining 5′ or 3′, intron or other control sequences from genomic libraries. One primer is synthesized to initiate extension in the antisense direction and the other is synthesized to extend sequence in the sense direction. Primers are used to facilitate the extension of the known sequence “outward” generating amplicons containing new, unknown nucleotide sequence for the region of interest. The initial primers are designed from the cDNA using OLIGO 4.06 primer analysis software (National Biosciences), or another appropriate program, to be 22-30 nucleotides in length, to have a GC content of 50% or more, and to anneal to the target sequence at temperatures about 68-72° C. Any stretch of nucleotides which would result in hairpin dimerizations is avoided. The original, selected cDNA libraries, or a human genomic library are used to extend the sequence, the latter is most useful to obtain 5′ upstream regions. If more extension is necessary or desired, additional sets of primers are designed to further extend the known region. By following the instructions for the XL-PCR kit (Perkin Elmer) and thoroughly mixing the enzyme and reaction mix, high fidelity amplification is obtained. Beginning with 40 pmol of each primer and the recommended concentrations of all other components of the kit, PCR is performed using the Peltier thermal cycler (PTC200; M. J. Research, Watertown, Mass.) and the following parameters:

Step 1 94° C. for 1 min (initial denaturation)

Step 2 65° C. for 1 min

Step 3 68° C. for 6 min

Step 4 94° C. for 15 sec

Step 5 65° C. for 1 min

Step 6 68° C. for 7 min

Step 7 Repeat step 4-6 for 15 additional cycles

Step 8 94° C. for 15 sec

Step 9 65° C. for 1 min

Step 10 68° C. for 7-15 min

Step 11 Repeat step 8-10 for 12 cycles

Step 12 72° C. for 8 min

Step 13 4° C. (and holding)

A 5-10 μl aliquot of the reaction mixture is analyzed by electrophoresis on a low concentration (about 0.6-0.8% agarose mini-gel to determine which reactions were successful in extending the sequence. Bands thought to contain the largest products are selected and removed from the gel. Further purification involves using a commercial gel extraction method such as the QIAQUICK DNA purification kit (QIAGEN). After recovery of the DNA, Klenow enzyme is used to trim single-stranded, nucleotide overhangs creating blunt ends which facilitate religation and cloning. After ethanol precipitation, the products are redissolved in 13 μl of ligation buffer, 1 μl T4-DNA ligase (15 units) and 1 μl T4 polynucleotide kinase are added, and the mixture is incubated at room temperature for 2-3 hours or overnight at 16° C. Competent E. coli cells (in 40 μl of appropriate media) are transformed with 3 μl of ligation mixture and cultured in 80 μl of SOC medium (Sambrook et al., supra). After incubation for one hour at 37° C., the whole transformation mixture is plated on Luria Bertani (LB)-agar (Sambrook et al., supra) containing 2× Carb. The following day, several colonies are randomly picked from each plate and cultured in 150 μl of liquid LB/2× Carb medium placed in an individual well of an appropriate, commercially-available, sterile 96-well microtiter plate. The following day, 5 μl of each overnight culture is transferred into a non-sterile 96-well plate and after dilution 1:10 with water, 5 μl of each sample is transferred into a PCR array. For PCR amplification, 18 μl of concentrated PCR reaction mix (3.3×) containing 4 units of rTth DNA polymerase, a vector primer, and one or both of the gene specific primers used for the extension reaction are added to each well. Amplification is performed using the following conditions:

Step 1 94° C. for 60 sec

Step 2 94° C. for 20 sec

Step 3 55° C. for 30 sec

Step 4 72° C. for 90 sec

Step 5 Repeat steps 2-4 for an additional 29 cycles

Step 6 72° C. for 180 sec

Step 7 4° C. (and holding)

Aliquots of the PCR reactions are run on agarose gels together with molecular weight markers. The sizes of the PCR products are compared to the original partial cDNAs, and appropriate clones are selected, ligated into plasmid, and sequenced.

VI. Labeling and Use of Hydridization Probes

Hybridization probes derived from SEQ ID NO:1 or SEQ ID NO:3are employed to screen cDNAs, genomic DNAs, or mRNAs. Although the labeling of oligonucleotides, consisting of about 20 base-pairs, is specifically described, essentially the same procedure is used with larger cDNA fragments. Oligonucleotides are designed using state-of-the-art software such as OLIGO 4.06 primer analysis software (National Biosciences, labeled by combining 50 pmol of each oligomer and 250 μCi of [.gamma.−32 P] adenosine triphosphate (Amersham) and T4 polynucleotide kinase (DuPont Nen(r), Boston, Mass.). The labelled oligonucleotides are substantially purified with SEPHADEX G-25 superfine resin column (Pharmacia & Upjohn). A portion containing 107 counts per minute of each of the sense and antisense oligonucleotides is used in a typical membrane based hybridization analysis of human genomic DNA digested with one of the following membranes (Ase I, BgI II, EcoRI, Pst I, Xba 1, or Pvu II; DuPont NEN(r)). The DNA from each digest is fractionated on a 0.7 percent agarose gel and transferred to nylon membranes (NYTRAN PLUS membrane, Schleicher & Schuell, Durham, N.H.). Hybrization is carried out for 16 hours at 40° C. To remove nonspecific signals, blots are sequentially washed at room temperature under increasingly stringent conditions up to 0.1×saline solution citrate (SSC) and 0.5% sodium dodecyl sulfate. After XOMAI AR Autoradiography film (Kodak Rochester, N.Y.) is exposed to the blots, or the blots are placed in a PHOSPHOIMA-GER (Molecular Dynamics, Sunnyvale, Calif.) for several hours, hybridization patterns are compared visually.

VII. Antisense Molecules

Antisense molecules to the pD52-encoding or pRA006 encoding sequence, or any part thereof, is used to inhibit in vivo or in vitro expression of naturally occurring pD52 or pRA006. Although use of antisense oligonucleotides, comprising about 20 base-pairs, is specifically described, essentially the same procedure is used with larger cDNA fragments. An oligonucleotide is used to inhibit expression of naturally occurring pD52 or pRA006. The complementary oligonucleotide is used either to inhibit transcription by preventing promoter binding to the upstream nontranslated sequence to translation of an pD52- or pRA006-encoding transcript by preventing the ribosome from binding.

VIII. Expression of pD52 or pRA006

Expression of pD52 or pRA006 is accomplished by subcloning the cDNAs into appropriate vectors and transforming the vectors into host cells. In this case, the cloning vector, PSPORT 1, previously used for the generation of the cDNA library is used to express pD52 or pRA006 in E. coli. Upstream of the cloning site, this vector contains a promoter for 62-galactosidase, followed by sequence containing the amino-terminal Met, and the subsequent seven residues of β-galactosidase. Immediately following these eight residues is a bacteriophage promoter useful for transcription and a linker containing a number of unique restriction sites. Induction of an isolated, transformed bacterial strain with IPTG using standard methods produces a fusion protein which consists of the first eight residues of β-galactosidase, about 5 to 15 residues of linker, and the full length protein. The signal residues direct the secretion of pD52 or pRA006 into the bacterial growth media which can be used directly in the following assay for activity.

IX. Production of pD52 or pRA006 Specific Antibodies

pD52 or pRA006 that is substantially purified using PAGE electrophoresis (Sambrook, supra), or other purification techniques, is used to immunize rabbits and to produce antibodies using standard protocols. The amino acid sequence deduced from SEQ ID NO:2 or SEQ ID NO:4 is analyzed using DNASTAR software (DNASTAR Inc) to determine regions of high immunogenicity and a corresponding oligopolypeptide is synthesized and used to raise antibodies by means known to those of skill in the art. Selection of appropriate epitopes, such as those near the C-terminus or in hydrophilic regions, is described by Ausubel et al. (supra), and others.

Typically, the oligopeptides are 15 residues in length, synthesized using an Applied Biosystems 431A peptide synthesizer 431A using fmoc-chemistry, and coupled to keyhole limpet hemocyanin (KLH, Sigma, St. Louis, Mo.) by reaction with N-maleimidobenzoyl-N-hydroxysuccinimide ester (MBS; Ausubel et al., supra). Rabbits are immunized with the oligopeptide-KLH complex in complete Freund's adjuvant. The resulting anti-sera are tested for antipeptide activity, for example, by binding the peptide to plastic, blocking with 1% BSA, reacting with rabbit antisera, washing, and reacting with radioiodinated, goat anti-rabbit IgG.

pD52 or pRA006 or biologically active fragments thereof are labeled with ¹²⁵I Bolton-Hunter reagent (Bolton et al. (1973) Biochem. J. 133:529). Candidate molecules previously arrayed in the wells of a multi-well plate are incubated with the labeled pD52 or pRA006, washed and any wells with labeled pD52 or pRA006 complex are assayed. Data obtained using different concentrations of pD52 or pRA006 are used to calculate values for the number, affinity, and association of pD52 or pRA006 with the candidate molecules. All publications and patents mentioned in the above specification are herein incorporated by reference.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. Use of a nucleic acid molecule of the pD52 or pRA006 gene family or a polypeptide encoded thereby or a fragment or variant of said nucleic acid molecule or said polypeptide or an antibody, an aptamer or an other receptor recognizing a nucleic acid molecule of the pD52 or pRA006 gene family or a polypeptide encoded thereby for the manufacture of a pharmaceutical agent.

2. The use of claim 1 wherein the nucleic acid molecule is a vertebrate D52 nucleic acid, particularly a human D52 nucleic acid (GenBank Acc. No. S 82081 or AAB 36476) or a human RA006 homolog nucleic acid (GenBank Accession No. CAB 53688), or a complementary sequence, or a fragment thereof or variant thereof.

3. The use of claim 1 or 2, wherein said nucleic acid molecule
(a) hybridizes at 22° C. in a solution containing 0.2×SSC and 0.1% SDS to the complementary strand of a nucleic acid molecule of SEQ ID NO:1 or SEQ ID NO:3 encoding the amino acid sequence of SEQ ID NO: 2 or SEQ ID NO:4,
(b) it is degenerate with respect to the nucleic acid molecule of (a),
(c) encodes a polypeptide which is at least 85%, preferably at least 90%, more preferably at least 95%, more preferably at least 98% and up to 99.6% identical to SEQ ID NO: 2 or SEQ ID NO: 4, or
(d) differs from the nucleic acid molecule of (a) to (c) by mutation and wherein said mutation causes an alteration, deletion, duplication or premature stop in the encoded polypeptide.

4. The use of any one of claims 1-3, wherein the nucleic acid molecule is a DNA molecule, particularly a cDNA or a genomic DNA.

5. The use of any one of claims 1-4, wherein the nucleic acid molecule encodes a pD52 protein or homolog protein that is expressed in the embryonic pancreatic epithelium, in islet cells of the pancreas, in adipocytes, and in intestinal crypt cells, or wherein the nucleic acid molecule encodes a pRA006 protein or homolog protein that is expressed in a subset of islet cells of the pancreas, the eye, as well as the central and peripheral nervous system.

6. The use of any one of claims 1-5, wherein said nucleic acid molecule is a recombinant nucleic acid molecule.

7. The use of claim 6, wherein said recombinant nucleic acid molecule is a vector, particularly an expression vector.

8. The use of any one of claims 1-5, wherein said polypeptide is a recombinant polypeptide.

9. The use of claim 8, wherein said polypeptide is a fusion polypeptide.

10. The use of any one of claims 1-7, wherein said nucleic acid molecule is selected from hybridization probes, primers and anti-sense oligonucleotides.

11. The use of any one of claims 1-10 for diagnostic applications.

12. The use of any one of claims 1-10 for therapeutic applications.

13. The use of any one of claims 1-12 for the manufacture of an agent for diagnosis, monitoring, prevention or treatment of metabolic disorders, particularly disorders associated with fat and/or carbohydrate metabolism, as well as neurodegenerative disorders.

14. The use of claim 13 for detecting and/or verifying, for the treatment, alleviation and/or prevention of an metabolic disorder or metabolic syndrome, such as pancreatic dysfunction (for example diabetes, hyperglycemia, and impaired glucose tolerance), obesity, adipositas, eating disorders (bulimia nervosa, anorexia nervosa), cachexia (wasting), and neurodegenerative disorders, and others, in cells, cell masses, organs and/or subjects.

15. Use of a nucleic acid molecule or a polypeptide encoded thereby or a fragment or variant of said nucleic acid molecule or said polypeptide or an antibody, an aptamer or an other receptor recognizing a nucleic acid molecule of the pD52 or pRA006 gene family or a polypeptide encoded thereby as defined in any one of claims 1-10 for monitoring and/or controlling the function of a gene and/or a gene product which is influenced and/or modified by a pD52 or pRA006 polypeptide.

16. Use of a nucleic acid molecule or a polypeptide encoded thereby or a fragment or variant of said nucleic acid molecule or said polypeptide or an antibody, an aptamer or an other receptor recognizing a nucleic acid molecule of the pD52 or pRA006 gene family or a polypeptide encoded thereby as defined in any one of claims 1-10 for identifying substances capable of interacting with a pD52 or pRA006 polypeptide.

17. A non-human transgenic animal exhibiting a modified expression of a pD52 or pRA006 polypeptide.

18. The animal of claim 17, wherein the expression of the pD52 or pRA006 polypeptide is increased and/or reduced.

19. A recombinant host cell exhibiting a modified expression of a pD52 or pRA006 polypeptide.

20. The cell of claim 19 which is a human cell.

21. A method of identifying a (poly)peptide involved in a metabolic disorder or metabolic syndrome, particularly in pancreatic dysfunction, in a mammal comprising the steps of
(a) contacting a collection of test (poly)peptides with a pD52 or pRA006 polypeptide or a fragment thereof under conditions that allow binding of said test (poly)peptide;
(b) removing test (poly)peptides which do not bind and
(c) identifying test (poly)peptides that bind to said pD52 or pRA006 polypeptide or the fragment thereof.

22. A method of screening for an agent which modulates the interaction of a pD52 or pRA006 polypeptide or a fragment thereof with a binding target/agent, comprising the steps of
(a) incubating a mixture comprising
(aa) a pD52 or pRA006 polypeptide or a fragment thereof;
(ab) a binding target/agent of said pD52 or pRA006 polypeptide or fragment thereof; and
(ac) a candidate agent under conditions whereby said pD52 or pRA006 polypeptide or fragment thereof specifically binds to said binding target/agent at a reference affinity;
(b) detecting the binding affinity of said pD52 or pRA006 polypeptide or fragment thereof to said binding target to determine an (candidate) agent-biased affinity; and
(c) determining a difference between (candidate) agent-biased affinity and the reference affinity.

23. A method of producing a composition comprising (poly)peptide identified by the method of claim 21 or the agent identified by the method of claim 22 with a pharmaceutically acceptable carrier and/or diluent.

24. The method of claim 23 wherein said composition is a pharmaceutical composition for preventing, alleviating or treating of metabolic disorder or metabolic syndrome, such as pancreatic dysfunction (for example diabetes, hyperglycemia, and impaired glucose tolerance), obesity, adipositas, eating disorders (bulimia nervosa, anorexia nervosa), cachexia (wasting), and neurodegenerative disorders, and other diseases and disorders.

25. Use of a (poly)peptide as identified by the method of claim 21 or of an agent as identified by the method of claim 23 for the preparation of a pharmaceutical composition for the treatment, alleviation and/or prevention of metabolic disorder or metabolic syndrome, such as pancreatic dysfunction (for example diabetes, hyperglycemia, and impaired glucose tolerance), obesity, adipositas, eating disorders (bulimia nervosa, anorexia nervosa), cachexia (wasting), and neurodegenerative disorders, and other diseases and disorders.

26. Use of a nucleic acid molecule of the pD52 or pRA006 gene family or of a fragment thereof for the preparation of a non-human animal which over- or underexpresses the pD52 or pRA006 gene product.

27. Kit comprising at least one of
(a) a pD52 or pRA006 nucleic acid molecule or a fragment thereof;
(b) a vector comprising the nucleic acid of (a);
(c) a host cell comprising the nucleic acid molecule of (a) or the vector of (b);
(d) a polypeptide encoded by the nucleic acid molecule of (a);
(e) a fusion polypeptide encoded by the nucleic acid molecule of (a);
(f) an antibody, an aptamer or another receptor the nucleic acid molecule of (a) or the polypeptide of (d) or (e) and
(g) an anti-sense oligonucleotide of the nucleic acid molecule of (a).


SEQUENCE LISTING (8 sequences)

SEQ ID NO: 1 - DNA, 2100 bp, chicken
ccgggtcgacccacgcgtccgttcagaaatcactttctggacggctgcatcgaatttaagagaggtgttggccaaatttgatgaggaggtacagtgagagccgggtgaagaaacgaggggcgaagaaagccccgtgctctggtgtggccgcccaaagagaagatgcctccttcagcagaagcagagggtggcaatactctctgagcttcctggcatggatctgtacgaggattacaagtcgccctttgatttcaatgcaggagtggacagaaactacctttacctttcaccgggcataaaccttactccacctgggtcaccaacgctgaccaagtctgggctgctgagaactgactctatacctgaggtcggagaggatgctgctgctacagttggcatgtcagaaactctctcagaagaagaacagaatgagctaagaaaagagcttgccaaggtggaagaggaaatccagacactctcacaagtgctagctgccaaagagaagcatctagcagaaatcaagagaaagctgggaattaactctttacaggaactaaagcagaatatcaccaaaagctggcaagatgttacgtcaactgcagcgtacaagaaaacatcagaaaccctgtctcaggcaggccagaaggcttctgctgcattttcctctgttggttcagtcataaccaagaagtttgaagatgtcaaaaattcttctactttcaaatctttcgaggaaaaagttgaaaccttaaagtctaaagtcggaggaagcaaacctgccagcggagactttggagaagttctgaactcagctgccaatgccagtgccacagaaacgattgcagaagagacacaggaggagacccactaagatccctgcctctgtcctgctacccactgccagatgctgcaagcaagaaccaagcacacttgtaacattctttgtgctctttccaccagatgtgcttttatttagcacgtagctgttttgccagattaactgattccaagcttgttgtgggttcagattatttttgaaactaaaatatgttggttgtttggggttttgtgtggggaggggaatttttatgcctttcaagcatttaaaggacatatcccttatttgaaggcaacctttaaaatatagcaatgaaaaactctttggagaagttaattatactgtacatacatgtggatacataattctttttgaatctgtaactactgttcaacttaatcattttgaatctaccgctgctgccaaactgaacctcgctacttcactcccttgtcattatttcagttaagtgttgcctgcatgggacaccagatttaggaggggcactgccagcttaaagaaaggcctttggcaggctttaaacaagttacttgtatcaggcttacagatgcgtgggaatgtactgtattaagagcttcttccaagagatgtgagtggggtaatgcataacatctagaacttaggactcgtctgtgggttatccaactaggaaggccctgactattagaagcaagatgcctattagtgctggtttagttcagttatgataacactgtcaacatagcattttgtacaaaacaatgggggaaggcgtactggttcttctctcaggcatcaaactgttttcaagtgctaatgttgttttccttgtagcctggccattgagaaaatctgccacatctgttattttctgtagctgtgaaggacctgtagcatccgtgccccgctcctgttaacacctaaaagctgtaccagtgtcgtgctgcagcactgctctcctccaggggctgctctcctgcctgttgctttatgccattgttctcttcagattctgttttctgtttctgacacaagatgtgggcgagatcataatttttggcaaatgttttcgtcgccgtgcttagaactggaaacaagctttgaaacagttgtgtgaaatctgtgctccttcaactgctgcagatgtagagaagaaaaaaggttccccacgcctttattagcatgtcagttgcagtgtacatatgctcaattacatgtccgaagttcaataaaaccagtatgtgagtaaaaaaaaaaaaaaa

SEQ ID NO: 2 - protein, 218 aa, chicken
MDLYEDYKSPFDFNAGVDRNYLYLSPGINLTPPGSPTLTKSGLLRTDSIPEVGEDAAATVGMSETLSEEEQNELRKELAKVEEEIQTLSQVLAAKEKHLAEIKRKLGINSLQELKQNITKSWQDVTSTAAYKKTSETLSQAGQKASAAFSSVGSVITKKFEDVKNSSTFKSFEEKVETLKSKVGGSKPASGDFGEVLNSAANASATETIAEETQEETH

SEQ ID NO: 3 - DNA, 1931 bp, chicken
gtcgacccacgcgtccgctctgctggccgccttccgtggagggggcgagacgggcgcagaccccgcagccgcgcgatggttgacatccccagcctcgtgaaaatcagcgtcgccctcaaaatccagcccaacgacggggcggtgtacttcaaggtggacgggcagcgtttcggccagaaccgcaccatcaagctgctcaccggggccaagtacaagattgaggtggccctccggcccggcaccgtgcaggcaacgacgatgggcatcgggggtgtcaatgtcccactggaagagaaatcgagggatgcacaagtggcctcttacacagggatctacgacacagaaggggtgccccacaccaagagtggggagagacagcccatccaggtcaacatgcagtttaacgacattggtgtttttgaaacagtctggcaagtcaaattctacaactaccacaaacgagatcactgccaatggggaaacagctttggcagtatagagtatgaatgcaaaccaaatgaaacacggagtcttatgtggatcaataaagagaccttccactgaacagaagtgaaaccaatactgctggacaatctccttaaagaaaaatctacccttttaaaccagcaagaagccttaccaaaggcatactgcaacgtcgctttgttccgtaaggtcccaataactcagctgtatgtacaggtggtgccattaaagtctttgcctattactgtatttcaagtttactatggcatctgcaagtctttatctataccctgggaatactgtaggctttaaggtgaagaacgttttattcctgggcagaaagtcatacaatcataatactatcataaccatttggatgatgcccactaaacagttccattagatgtataaacctttgtgaagtgttttgctttgttgcttaaaatttggatgcaacatcacattggtgatctccatatctatatttgtttgctccttcatcagtgtgcaccttcaataattttgtctaaacatcatctcagtggacagaaaaaagtgttaattatggatgtctgtaggtttagacaaatggcagctactgcactgttgatattgtgctttcatatcaaggatttggaaattcactctcctttctgttgagtgctgtaagataccctgtgataaatatcctattttgtgagatttggtaaattagcactctttgatgttaagtttcccagtctctgaaatagctttaggaaatggtataaaacatgctaacctgaatgaaagaaaagcaataaatcagttgtggtactaacaacatctaacagcctggctagcatctatgttaagatcatgtagtcttattgaaagagatagcagccatcctcaatttagttcttagaatatgagttttgataataaactccaggtttttttcaatgaaatattgctcaaaatcattgaaatcccataaatcagctttgttaagcctgaccactacattataccaaaatctataggatccccaaacaagaaaaactcagcatagtgacaaaactacagatctcagtgcatgcttatacatgcactgtgctctttacaacaaaggaaagcaggtcagtatatgaatagatacaactaaaggtgctacctaaacactaggtcagtgtttaacacatacagataacaacaacaaaaacacacaaaaagacatcttttctttcagtaaataaagcagggaagcaactatactgtcccatctcctacttgggctaaagtaagctgtcagttcttgcatacctgttacttcctttgtgaataatttgtaaattgtgtatgtaaatggtaaccatgacttttgatctttgtaataaaggatttcaacagttaaaaaaaaaaaaaaaaaaaaaaagggcggccgctctagaggatccaagctt

SEQ ID NO: 4 - protein, 162 aa, chicken
MVDIPSLVKISVALKIQPNDGAVYFKVDGQRFGQNRTIKLLTGAKYKIEVALRPGTVQATTMGIGGVNVPLEEKSRDAQVASYTGIYDTEGVPHTKSGERQPIQVNMQFNDIGVFETVWQVKFYNYHKRDHCQWGNSFGSIEYECKPNETRSLMWINKETFH

SEQ ID NO: 5 - protein, 251 aa, quails
MVAVLSELPGMDLYEDYKSPFDFNAGVDRTYLYLSPGINLTPPGSPTLTKSGLLRTDSIPEVGEDAAATVGMSETLSEEEQNELRKELAKVEEEIQTLSQVLAAKEKHLAEIKRKLGINSLQELKQNITKSWQDVTSTAAYKKTSETLSQAGQKASAAFSSVGSVLSKKFEDVKLQAFSHSFSIRSIQHSISMPIMRNSPTFKSFEEKVETLKSKVGGSKPASGDFGEVLNSAANASATETIAEETQEETH

SEQ ID NO: 6 - protein, 185 aa, Mouse
MDRGEQGLLKTEPVAEEGEDAVTMLSAPEALTEEEQEELRRELTKVEEEIQTLSQVLAAKEKHLAELKRKLGISSLQEFKQNIAKGWQDVTATNAYKKTSETLSQAGQKASAAFSSVGSVITKKLEDVKNSPTFKSFEEKVENLKSKVGGAKPAGGDFGEVLNSTANATSTMTTEPPPEQMTESP

SEQ ID NO: 7 - protein, 184 aa, Human
MDRGEQGLLRTDPVPEEGEDVAATISATETLSEEEQEELRRELAKVEEEIQTLSQVLAAKEKHLAEIKRKLGINSLQELKQNIAKGWQDVTATSAYKKTSETLSQAGQKASAAFSSVGSVITKKLEDVKNSPTFKSFEEKVENLKSKVGGTKPAGGDFGEVLNSAANASATTTEPLPEKTQESL

SEQ ID NO: 8 - protein, 161 aa, Human
DLPGLVRLSIALRTQPNDGPVFYKVDGQRFGQNRTIKLLTGSSYKVEVKIKPSTLQVENISIGGVLVPLELKSKEPDGDRVVYTGTYDTEGVTPTKSGERQPIQITMPFTDIGTFETVWQVKFYNYHKRDHCQWGSPFSVIEYECKPNETRSLMWVNKESF